(19) United States
(12) Patent Application Publication
(10) Pub. No.: US 2026/0295097 A1
(43) Pub. Date: Oct. 1, 2026

(54) GAS-FILLED MICROVESICLES WITH PERFLUORO OLEFIN

(71) Applicant: Bracco Suisse SA, Cadempino (CH)

(72) Inventors: Philippe BUSSAT, Pers-Jussy (FR); Samir CHERKAOUI, Feigeres (FR)

(73) Assignee: Bracco Suisse SA, Cadempino (CH)

(21) Appl. No.: 19/139,638

(22) PCT Filed: Dec. 21, 2023
(86) PCT No.: PCT/EP2023/087498

§ 371 Date: Jun. 16, 2025

(30) Foreign Application Priority Data
Dec. 21, 2022 (EP) 22215345

(51) Int. Cl. A61K 49/22

(52) U.S. Cl. CPC A61K 49/223

(57) ABSTRACT

The present invention relates to a suspension of gas-filled microvesicles comprising an outer layer of stabilizing material surrounding a gaseous core, said gaseous core comprising a perfluoropentene.

### TECHNICAL FIELD

The present invention relates to a suspension of gas-filled microvesicles comprising a perfluorinated olefin in their gaseous core.

### BACKGROUND OF THE INVENTION

Suspensions of gas-filled microvesicles (typically dispersed in a suitable physiologically acceptable solution) are known since decades for contrast-enhanced Ultrasound imaging (CEUS) and more recently also for therapeutic treatments. The gas is typically entrapped or encapsulated in a layer of stabilizing material comprising, for instance, emulsifiers, oils, thickeners or sugars. Gas-filled microvesicles are generally referred to in the art with various terminologies, depending typically on the stabilizing material employed for their preparation; these terms include, for instance, “microspheres”, “microbubbles”, “microcapsules” or “microballoons”, globally referred to herein as “gas-filled microvesicles” (in brief: “microvesicles” or “GfM”). As used in the art, the term GfM refers, to microvesicles of either micrometric size (e.g. from 1 to 10, typically 2-8 microns) or nanometric size (e.g. 50 up to 1000 nanometers, typically 100-400 nanometers).

Suspensions of GfM can be produced according to various manufacturing methods.

Some of these methods, see (e.g. WO94/09829 or WO2004/069284) entail the initial preparation of solutions or emulsions comprising the desired stabilized materials, in admixture with a freeze-drying protecting component. The preparation is then distributed into vials which are subjected to a freeze-drying process to remove water and/or solvents. The headspace of the vials is then filled with a suitable gas (e.g. a fluorinated gas) and sealed. Before use, an aqueous suspension of microbubbles is easily prepared by introducing a suitable liquid into the vial (e.g. saline, glucose solution, etc.) and gently shaking the vial to dissolve the freeze-dried product and form the microbubbles.

Other preparation methods include the preparation of aqueous suspensions of stabilizing material in contact with a suitable gas (in the headspace of the vial containing the suspension); the GfM are prepared before use by vigorous shaking of the vial.

More recently, microfluidic techniques (see e.g. WO2020/260423) allow to manufacture suspension of GfM of calibrated size.

Fluorinated gases, including sulfur hexafluoride, perfluoropropane and perfluorobutane have been the gases of choice for the preparation of gas-filled microbubbles for ultrasound medical applications because of their low solubility and low diffusivity.

However, such perfluorinated gases, while providing very stable microbubbles suspensions, have a relatively long atmospheric lifetime and high global-warming potential (GWP). GWP is a term used to describe the relative potency of a single greenhouse gas, taking account of how long it remains active in the atmosphere. The global-warming potentials (GWPs) currently used are those calculated over 100 years. Carbon dioxide is taken as the gas of reference and given a 100-year GWP of 1. For instance, SF₆ has a GWP of 23500, C₄F₁₀ of 9200 and C₃F₈ of 8900 (see e.g. Myhre, G., D. Shindell, F.-M. Bréon, W. Collins, J. Fuglestvedt, J. Huang, D. Koch, J.-F. Lamarque, D. Lee, B. Mendoza, T. Nakajima, A. Robock, G. Stephens, T. Takemura and H. Zhang, 2013: Anthropogenic and Natural Radiative Forcing. In: Climate Change 2013: The Physical Science Basis. Contribution of Working Group I to the Fifth Assessment Report of the Intergovernmental Panel on Climate Change [Stocker, T. F., D. Qin, G.-K. Plattner, M. Tignor, S. K. Allen, J. Boschung, A. Nauels, Y. Xia, V. Bex and P. M. Midgley (eds.)]. Cambridge University Press, Cambridge, United Kingdom and New York, NY, USA.)

The Applicant has now found a suitable class of compounds with relatively low GWP values which can advantageously replace the current perfluorinated compounds in the preparation of gas-filled microvesicles.

### SUMMARY OF THE INVENTION

An aspect of the present invention relates to a suspension of gas-filled microvesicles, said gas-filled microvesicles comprising an outer layer of stabilizing material surrounding a gaseous core, wherein said gaseous core comprises a perfluoro pentene. Preferably the compound is perfluoro-2-pentene.

The perfluorinated compound is preferably admixed with air or nitrogen, preferably nitrogen. The volume percentage of the perfluorinated compound is of 10% or higher. In certain embodiments, the amount of perfluorinated compound may be of 20%, 30%, 35%, 40%, 50% or higher, up to e.g. 70%, preferably up to 60%.

Another aspect of the invention relates to a suspension of gas-filled microvesicles in a physiologically acceptable liquid wherein said microvesicles are as above defined.

A further aspect of the invention relates to a vial comprising:
a precursor of gas-filled microvesicles in the form of a freeze-dried product comprising a stabilizing material capable of forming a stabilizing layer for said microvesicles and a freeze-drying protecting component; and
a perfluoro pentene.

### DETAILED DESCRIPTION OF THE INVENTION

### GfM Components

Stabilizing materials suitable for forming the outer layer of the GfMs include amphiphilic materials, such as phospholipids, biodegradable polymers or biodegradable water-insoluble lipids (such as those described, for instance, in U.S. Pat. Nos. 5,711,933 and 6,333,021), proteins (e.g. albumin, haemoglobin, as described for instance U.S. Pat. No. 4,276,885 or EP 0324938).

Preferably the stabilizing material is an amphiphilic material.

According to a preferred embodiment, amphiphilic materials useful for forming the stabilizing layer comprise a phospholipid. Phospholipids, as other amphiphilic molecules, are generally capable of forming a stabilizing film of material (typically in the form of a mono-molecular layer) at the gas-water boundary interface in the final gas-filled microvesicles suspension, these materials are also referred to in the art as “film-forming” materials.

Phospholipids typically contain at least one phosphate group and at least one, preferably two, lipophilic long-chain hydrocarbon group.

Examples of suitable phospholipids include esters of glycerol with one or preferably two (equal or different) residues of fatty acids and with phosphoric acid, wherein the phosphoric acid residue is in turn bound to a hydrophilic group, such a, for instance, choline (phosphatidylcholines-PC), serine (phosphatidylserines-PS), glycerol (phosphatidylglycerols-PG), ethanolamine (phosphatidylethanolamines-PE), inositol (phosphatidylinositol). Esters of phospholipids with only one residue of fatty acid are generally referred to in the art as the “lyso” forms of the phospholipid or “lysophospholipids”. Fatty acids residues present in the phospholipids are in general long chain aliphatic acids, typically containing from 12 to 24 carbon atoms, preferably from 14 to 22; the aliphatic chain may contain one or more unsaturations or is preferably completely saturated. Examples of suitable fatty acids included in the phospholipids are, for instance, lauric acid, myristic acid, palmitic acid, stearic acid, arachidic acid, behenic acid, oleic acid, linoleic acid, and linolenic acid. Preferably, saturated fatty acids such as myristic acid, palmitic acid, stearic acid and arachidic acid are employed.

Further examples of phospholipid are phosphatidic acids, i.e. the diesters of glycerol-phosphoric acid with fatty acids; sphingolipids such as sphingomyelins, i.e. those phosphatidylcholine analogs where the residue of glycerol diester with fatty acids is replaced by a ceramide chain; cardiolipins, i.e. the esters of 1,3-diphosphatidylglycerol with a fatty acid; glycolipids such as gangliosides GM1 (or GM2) or cerebrosides; glucolipids; sulfatides and glycosphingolipids.

As used herein, the term “phospholipid(s)” includes either naturally occurring, semisynthetic or synthetically prepared compounds that can be employed either alone or as mixtures.

Examples of naturally occurring phospholipids are natural lecithins (phosphatidylcholine (PC) derivatives) such as, typically, soya bean or egg yolk lecithins.

Examples of semisynthetic phospholipids are the partially or fully hydrogenated derivatives of the naturally occurring lecithins. Preferred phospholipids are fatty acids di-esters of phosphatidylcholine, ethylphosphatidylcholine, phosphatidylglycerol, phosphatidic acid, phosphatidylethanolamine, phosphatidylserine, phosphatidylinositol or of sphingomyelin.

Examples of preferred phospholipids are, for instance, dilauroyl-phosphatidylcholine (1,2-Dilauroyl-sn-glycero-3-phosphocholine, DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), dibehenoyl-phosphatidylcholine (DBPC), 1,2 Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroyl-phosphatidylglycerol (DLPG) and its alkali metal salts, diarachidoylphosphatidyl-glycerol (DAPG) and its alkali metal salts, dimyristoylphosphatidylglycerol (DMPG) and its alkali metal salts, dipalmitoylphosphatidylglycerol (DPPG) and its alkali metal salts, distearoylphosphatidylglycerol (DSPG) and its alkali metal salts, dioleoyl-phosphatidylglycerol (DOPG) and its alkali metal salts, dimyristoyl phosphatidic acid (DMPA) and its alkali metal salts, dipalmitoyl phosphatidic acid (DPPA) and its alkali metal salts, distearoyl phosphatidic acid (DSPA), diarachidoylphosphatidic acid (DAPA) and its alkali metal salts, dimyristoyl-phosphatidylethanolamine (DMPE), dipalmitoylphosphatidylethanolamine (DPPE), distearoyl phosphatidyl-ethanolamine (DSPE), dioleylphosphatidyl-ethanolamine (DOPE), diarachidoylphosphatidyl-ethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), dioleoyl-phosphatidylinositol (DOPI).

Suitable phospholipids further include phospholipids modified by linking a hydrophilic polymer, such as polyethyleneglycol (PEG) or polypropyleneglycol (PPG), thereto. Preferred polymer-modified phospholipids include “pegylated phospholipids”, i.e. phospholipids bound to a PEG polymer. Examples of pegylated phospholipids are pegylated phosphatidylethanolamines (“PE-PEGs” in brief) i.e. phosphatidylethanolamines where the hydrophilic ethanolamine moiety is linked to a PEG molecule of variable molecular weight (e.g. from 300 to 20000 daltons, preferably from 500 to 5000 daltons), such as DPPE-PEG (or DSPE-PEG, DMPE-PEG, DAPE-PEG or DOPE-PEG). For example, DPPE-PEG5000 refers to DPPE having attached thereto a PEG polymer having a mean average molecular weight of about 5000. An example of DPPE-PEG5000 is the methoxy terminated PEG derivative 1,2-dipalmitoyl-sn-glycero-3-phosphoethanolamine-N-[methoxy (polyethylene glycol)-5000].

In an embodiment the phospholipids may bear a reactive moiety which may then be reacted with a corresponding reactive moiety bearing a suitable active component (e.g. targeting ligand), in order to bind said active component to the microvesicle. Examples of suitable reactive moieties include, for instance, reactive groups capable of reacting with an amino group bound to an active component such as isothiocyanate groups (that will form a thiourea bond), reactive esters (to form an amide bond), aldehyde groups (for the formation of an imine bond to be reduced to an alkylamine bond); reactive groups capable of reacting with a thiol group bound to an active component, such as haloacetyl derivatives or maleimides (to form a thioether bond); reactive groups capable of reacting with a carboxylic group bound to an active component, such as amines or hydrazides (to form amide or alkylamide bonds). Preferably, the amphiphilic compound bearing the reactive moiety is a lipid bearing a hydrophilic polymer, such as those previously mentioned, preferably a pegylated phospholipid, e.g. DPPE-PEG2000 or DSPE-PEG2000, such as DPPE-PEG2000-maleimide DSPE-PEG2000-maleimide.

Particularly preferred phospholipids are DBPC, DAPC, DSPC, DPPC, DMPA, DPPA, DSPA, DMPG, DPPG, DSPG, DMPS, DPPS, DSPS and Ethyl-DSPC. Most preferred are DPPG, DPPS and DSPC.

Mixtures of phospholipids can also be used, such as, for instance, mixtures of DPPE and/or DSPE (including pegylated derivates), DPPC, DSPC and/or DAPC with DSPS, DPPS, DSPA, DPPA, DSPG, DPPG, Ethyl-DSPC and/or Ethyl-DPPC.

The phospholipids can conveniently be used in admixture with any other compound, preferably amphiphilic. For instance, lipids such as cholesterol, ergosterol, phytosterol, sitosterol, lanosterol, tocopherol, propyl gallate or ascorbyl palmitate, fatty acids such as myristic acid, palmitic acid, stearic acid, arachidic acid and derivatives thereof or butylated hydroxytoluene and/or other non-phospholipid (amphiphilic) compounds can optionally be added to one or more of the foregoing phospholipids, e.g. in proportions preferably below 50% by weight, more preferably up to 25% or lower. Particularly preferred as additional compound in admixture with phospholipids are fatty acids. Fatty acids useful in a composition according to the invention, which can be either saturated or unsaturated, comprise a C₁₀-C₂₄, aliphatic chain terminated by a carboxylic acid moiety, preferably a C₁₄-C₂₂ and more preferably a C₁₆-C₂₀ aliphatic chain. Examples of suitable saturated fatty acids include capric (n-decanoic), lauric (n-dodecanoic), myristic (n-tetradecanoic), palmitic (n-hexadecanoic), stearic (n-octadecanoic), arachidic (n-eicosanoic), behenic (n-docosanoic) and n-tetracosanoic acid. Preferred saturated fatty acids are myristic, palmitic, stearic and arachidic acid, more preferably palmitic acid. Examples of unsaturated fatty acids comprise myristoleic (cis-9-tetradecenoic), palmitoleic (cis-9-hexadecenoic), sapienic (cis-6-hexadecenoic), oleic (cis-9-octadecenoic), linoleic (cis-9,12-octadecadienoic), linolenic (cis-9,12,15-octadecatrienoic), gondoic (cis-11-eicosenoic), cis-11,14-eicosadienoic, cis-5,8,11-eicosatrienoic, cis-8,11,14-eicosatrienoic, cis-11,14,17-eicosatrienoic, arachidonic (cis-8,11,14,17-eicosatetraenoic) and erucic (cis-13-docosenoic) acid.

According to an embodiment, the mixture of amphiphilic materials comprises a mixture of DSPC, DPPG and palmitic acid.

According to an alternative embodiment, said amphiphilic material comprises a mixture of DSPC, DPPE-PEG5000 and palmitic acid, optionally further comprising a targeting ligand.

Compositions and GfMs according to the invention may optionally comprise a targeting ligand, as described for instance in WO2020/229642.

### Method of Manufacturing

A suitable method for preparing injectable suspensions of gas-filled microvesicles comprises the reconstitution, in the presence of a suitable gas or gaseous mixture, of a freeze-dried product comprising a stabilizing material (capable of forming a stabilizing outer layer) with an aqueous carrier. The freeze-dried product is typically obtained by freeze-drying a liquid mixture comprising said amphiphilic material and a freeze-drying protecting component in a suitable solvent. The liquid mixture which undergoes the freeze-drying process can be obtained according to methods known in the art, as disclosed e.g. in WO94/09829 or WO2004/069284.

For instance, according to the process disclosed by WO94/09829, the amphiphilic material is dispersed into an organic solvent (e.g. tertiary butanol, dioxane, cyclohexanol, tetrachlorodifluoro ethylene or 2-methyl-2-butanol) together with a suitable freeze-drying protecting component. The dispersion containing the amphiphilic material and the freeze-drying protecting component is then subjected to freeze-drying to remove the organic solvent thus obtaining a freeze-dried product.

According to the alternative process disclosed in WO2004/069284, a composition comprising an amphiphilic material may be dispersed in an emulsion of water with a water immiscible organic solvent under agitation, preferably in admixture with a freeze-drying protecting component. The so obtained (micro) emulsion, which contains microdroplets of solvent surrounded and stabilized by the amphiphilic material, is then freeze-dried according to conventional techniques to obtain a freeze-dried material, which can then be used for preparing a suspension of gas-filled microvesicles.

As defined herein, a freeze-drying protecting component is a compound with cryoprotective and/or lyoprotective effect. Suitable freeze-drying protecting components include, for instance, carbohydrates, e.g. a mono-di- or poly-saccharide, such as sucrose, maltose, trehalose, glucose, lactose, galactose, raffinose, cyclodextrin, dextran, chitosan and its derivatives (e.g. carboxymethyl chitosan, trimethyl chitosan); polyols, e.g. sugar alcohols such as sorbitol, mannitol or xylitol; or hydrophilic polymers, e.g. polyoxyalkyleneglycol such as polyethylene glycol (e.g. PEG2000, PEG4000 or PEG8000) or polypropylenglycol. According to an embodiment said freeze-drying protecting component is polyethylene glycol, preferably PEG4000.

For the freeze-drying process, the liquid mixture containing the amphiphilic material and the freeze-drying protecting component (obtained e.g. according to either of the previously illustrated manufacturing processes), is typically sampled into glass vials (e.g. DIN4R, DIN8R or DIN20R) which are loaded into a freeze-dryer.

The freeze-drying process generally includes an initial step where the vials are rapidly deep-cooled (e.g. at temperatures of from −35° C. to −70° C.) to freeze the liquid(s) of the mixture and then subjected to vacuum (e.g. 0.1-0.8 mbar); during this step (primary drying), the substantial totality of the frozen liquid(s) (e.g. water and/or solvents) is removed by sublimation, typically up to about 95% of the total amount of liquid, preferably up to about 99%. After the primary drying, residual liquid (including possible interstitial water) can be further removed during the secondary drying, which is typically conducted at a temperature higher than room temperature, under vacuum (preferably by maintaining the same vacuum applied during the primary drying). The temperature during the secondary drying is preferably not higher than 35° C. The secondary drying can be stopped when the residual content of the liquid(s) reaches a desired minimum value, e.g. less than 3% (preferably less than 1%) by weight of water with respect to the total mass of residual freeze-dried product, or e.g. less than 0.01% by weight, preferably less than 0.08%, for residual solvent(s).

After completion of the freeze-drying process, the freeze-dried product may undergo an optional additional thermal treatment, typically under ambient pressure, as described for instance in WO2020/229642. Preferably the thermal treatment is performed on the sealed vial, after saturating the headspace of the vials with a suitable gas or gaseous mixture as defined herein and then stoppering (e.g. with a rubber, such as butyl rubber, stopper) and sealing (e.g. with a metal, such as aluminium, crimp seal) the vials. In this case, the vials are preferably removed from the freeze-drier and introduced in a suitable oven for the thermal treatment. Alternatively, such thermal treatment can be performed on the open vial (which are preferably kept into the freeze-dryer), which are then saturated with the gas or gaseous mixture and then stoppered/sealed.

### Perfluoro Pentenes

Suitable perfluoro pentenes for preparing GfM according to the invention include perfluoro-1-pentene, perfluoro-2-pentene, or mixtures thereof. Preferably the perfluoro pentene is perfluoro-2-pentene.

As observed by the Applicant, GfM comprising perfluoro pentenes in their gaseous core generally show improved pressure resistance properties with respect to GfM comprising lower molecular weight perfluoro olefins (e.g. C₄F₈). More surprisingly (as shown in the examples) the Applicant further observed that GfM comprising perfluoro pentenes in their gaseous core also show improved pressure resistance properties with respect to GfM comprising higher molecular weight perfluoro olefins (e.g. C₆F₁₂). Among the perfluoro pentenes, it has been found that GfM with perfluoro-2-pentene has an unexpectedly higher resistance to pressure, for instance with respect to GfM with perfluoro-1-pentene.

Preferably the perfluoro pentene is admixed with another gas, typically a non-fluorinated gas, such as air, oxygen, carbon dioxide or nitrogen, preferably nitrogen.

While the admixing of perfluorinated gases with increasing amounts of a non-fluorinated gas (e.g. nitrogen) may progressively reduce the pressure resistance of the GfM, it has nevertheless the advantage of reducing the total amount of fluorinated gas employed for the preparation of GfM. In addition, the admixing with a second gas (typically nitrogen, having e relatively low boiling point) has the advantage that perfluoro olefins having a boiling point close to or slightly higher than room temperature (such as perfluoro pentene) can be used for the preparation of the GfM. For instance, gaseous mixture containing 50% (v/v) of perfluoropentenes (having boiling points of 26° C.-29° C.) in admixture with nitrogen can be prepared. Notably, GfM containing relatively low percentages of perfluoro olefins (e.g. 20% by volume of perfluoropentene and 80% nitrogen) retain relatively high values of pressure resistance.

As used herein, the “pressure resistance” of GfMs is defined by the Pc50 parameter measured on a suspension of GfMs; as known, an increasing overpressure applied on a suspension of GfMs results in the progressive reduction of the population of microvesicles with respect to the initial one (measured at atmospheric pressure), due to the collapse of the microvesicles. As explained in detail in the experimental part, the Pc50 of a suspension of gas-filled microvesicles identifies the value of applied overpressure (with respect to atmospheric pressure) at which the absorbance of the suspension drops to half of the absorbance of the suspension measured at atmospheric pressure. Reduction of the absorbance of a suspension of GfMs is related to the reduction of the initial population of gas-filled microvesicles, whereby the initially milky suspension (typical of high concentration of microvesicles) becomes more and more transparent under increasing pressure (reduced concentration of GfMs). The higher the Pc50 values, the higher the resistance to pressure (e.g. blood pressure or ultrasound pressure) of microvesicles, and typically the longer the circulation time of the GfMs once administered.

### Use of GfM

The suspension of GfM according to the invention may be used in a variety of diagnostic and/or therapeutic techniques, including in particular ultrasound.

An aspect of the invention thus relates to the use in a method of diagnosing of a suspension of microvesicles according to the invention.

Diagnostic methods include any method where the use of the gas-filled microvesicles allows enhancing the visualisation of a portion or of a part of an animal (including humans) body, including imaging for preclinical and clinical research purposes. A variety of imaging techniques may be employed in ultrasound applications, for example including fundamental and harmonic B-mode imaging, pulse or phase inversion imaging and fundamental and harmonic Doppler imaging; if desired three-dimensional imaging techniques may be used.

Microvesicles according to the invention may typically be administered in a concentration of from about 0.01 to about 1.0 μL of gas per kg of patient, depending e.g. on their respective composition, the tissue or organ to be imaged and/or the chosen imaging technique. This general concentration range may of course vary depending on specific imaging applications, e.g. when signals can be observed at very low doses such as in colour Doppler or power pulse inversion.

In an embodiment said method of diagnosing comprises
(i) administering to a patient a suspension of gas-filled microvesicles according to the invention; and
(ii) detecting an ultrasound signal from a region of interest in said patient.

Another aspect of the invention relates to the use in a method of therapeutic treatment of a suspension of microvesicles according to the invention.

Therapeutic techniques include any method of treatment (as above defined) of a patient which comprises the combined use of ultrasounds and gas-filled microvesicles either as such (e.g. in ultrasound mediated thrombolysis, high intensity focused ultrasound ablation, blood-brain barrier permeabilization, immunomodulation, neuromodulation, radiosensitization) or in combination with a therapeutic agent (i.e. ultrasound mediated delivery, e.g. for the delivery of a drug or bioactive compound to a selected site or tissue, such as in tumor treatment, gene therapy, infectious diseases therapy, metabolic diseases therapy, chronic diseases therapy, degenerative diseases therapy, inflammatory diseases therapy, immunologic or autoimmune diseases therapy or in the use as vaccine), whereby the presence of the gas-filled microvesicles may provide a therapeutic effect itself or is capable of enhancing the therapeutic effects of the applied ultrasounds, e.g. by exerting or being responsible to exert a biological effect in vitro and/or in vivo, either by itself or upon specific activation by various physical methods (including e.g. ultrasound mediated delivery).

Microvesicles according to the invention can typically be administered for therapeutic purposes in a concentration of from about 0.01 to about 5.0 μL of gas per kg of patient, depending e.g. from their respective composition, the type of subject under treatment, the tissue or organ to be treated and/or the therapeutic method applied.

In an embodiment said method of ultrasound therapeutic treatment comprises:
(i) administering to a patient a suspension of gas-filled microvesicles according to the invention;
(ii) identifying a region of interest in said patient to be submitted to a therapeutic treatment, said region of interest comprising said suspension of gas-filled microvesicles; and
(iii) applying an ultrasound beam for therapeutically treating said region of interest;
whereby said ultrasound therapeutic treatment is enhanced by the presence of said suspension of gas-filled microvesicles in said region of interest.

In a further embodiment, the suspension of gas-filled microvesicles of the invention may be advantageously used in a method for separating cells, typically by buoyancy (also known as buoyancy-activated cell sorting, “BACS”). The method can be useful for separating a desired type of cells from other cells in a physiological liquid (e.g. blood or plasma). In an embodiment, the separation method comprises labelling a desired cell to be separated with a suitable labelled antibody capable of binding to a specific (and selective) receptor on said cell. The microvesicles of the invention are then added to the suspension of cells to be separated (including those bearing the labelled antibody); once admixed to the suspension of cells, the microvesicles associate through the ligand with the labelling residue bound to antibody/cell construct thus allowing separation of the cells by buoyancy (see e.g. WO 2017/117349). For instance, the labelled antibody is a biotinylated antibody, where the biotin residue is capable of associating with a respective moiety, such as for instance an avidin, neutravidin or streptavidin residue on a gas-filled microvesicles. The improved resistance to pressure allows using the microvesicles of the invention in a wide variety of methods for separating cells.

The following examples will help to further illustrate the invention.

### EXAMPLES

### Materials

DSPC: 1,2-distearoyl-sn-glycero-3-phosphocholine
DPPG-Na: 1,2-dipalmitoyl-sn-glycero-3-phospho-(1′-rac-glycerol) (sodium salt)
DPPE-PEG5000:1,2-dipalmitoyl-sn-glycero-3-phosphoethanolamine-N-[methoxy (polyethylene glycol)-5000] (ammonium salt)
PEG4000=Polyethyleneglycol (MW=4000 g/mol)
Palmitic acid
SF₆: Sulphur hexafluoride
C₄F₁₀: Prefluoro butane
C₃F₆: Perfluoropropene
1-C₄F₈: perfluoro-1-butene
2-C₄F₈: perfluoro-2-butene
1-C₅F₁₀: perfluoro-1-pentene
2-C₅F₁₀: perfluoro-2-pentene
1-C₆F₁₂: perfluoro-1-hexene
m-2-C₆F₁₂: perfluoro (4-methyl-2-pentene)

### Example 1

Preparation of Vials Containing a Freeze-Dried Product in Contact with Various Gases (Preparations C1 and 1a-1o)

The procedure illustrated in the working examples of WO 94/09829 was used for preparing a first batch of several vials containing a freeze-dried product in contact with a gaseous headspace. Briefly, DSPC, DPPG-Na and palmitic acid in a weight ratio of 4.75/4.75/1 were first dissolved in hexane/ethanol (8/2, v/v) at a concentration of about 5 g/L and the solvents were evaporated under vacuum. The solid residue was admixed with PEG4000 in a weight ratio of about 0.017:1, the mixture was dissolved in tert-butanol at around 60° C. and the clear solution was used to fill respective DIN8R vials (with a corresponding volume containing about 25 mg of the mixture). The vials were then rapidly cooled at −45° C. and then subjected the vacuum for removing the frozen solvent by sublimation. The temperature was then raised above room temperature and the remaining solvent was evaporated, down to a final amount of less than 0.5% by weight. At the end of the freeze-drying process, the ambient of the freeze-dryer was saturated with SF₆ at atmospheric pressure and the vials were stoppered and sealed. A thermal post-treatment, as described in WO2020/229642 was performed by heating the vials at about 45° C. for about 16 hours.

The vials were then divided in different groups of about 3 vials each.

The first group (C1, comparative) was kept as such, while in the other groups the headspace of the vials was replaced with perfluoro olefins or mixtures of perfluoro olefin with nitrogen (v/v), as defined in table 1:

TABLE 1
Composition of headspace in vials

| Preparation | Composition of headspace (vol) |
|---|---|
| C1 (comp) | 100% SF₆ |
| 1a | 100% C₃F₆ |
| 1b | 100% 2-C₄F₈ |
| 1c | 100% 1-C₄F₈ |
| 1d | 50% C₃F₆ + 50% N₂ |
| 1e | 50% 2-C₄F₆ + 50% N₂ |
| 1f | 50% 2-C₅F₁₀ + 50% N₂ |
| 1g | 20% C₃F₆ + 80% N₂ |
| 1h | 20% 2-C₄F₈ + 80% N₂ |
| 1i | 20% 1-C₄F₈ + 80% N₂ |
| 1j | 20% 2-C₅F₁₀ + 80% N₂ |
| 1k | 20% 1-C₅F₁₀ + 80% N₂ |
| 1l | 10% 1-C₆F₁₂ + 90% N₂ |
| 1m | 20% 1-C₆F₁₂ + 80% N₂ |
| 1n | 20% m-2-C₆F₁₂ + 80% N₂ |
| 1o | 35% m-2-C₆F₁₂ + 65% N₂ |

### Example 2

Preparation of Vials Containing a Freeze-Dried Product in Contact with Various Gases (Preparations C2 and 2a-2f)

The procedure illustrated in the working examples of WO2004/069284 was used for preparing a second batch of several vials containing a freeze-dried product in contact with a gaseous headspace. Briefly, an emulsion of cyclooctane and water (about 1.5/100 v/v) containing about 90 mg/L of DSPC, 7 mg/L of palmitic acid, 60 mg/L of DPPE-PEG5000 and 100 g/L of PEG4000 is prepared (Megatron MT3000, Kinematica; 10′000 rpm) and sampled into DIN8R vials (about 0.75 mL/vial).

The vials were cooled at −50° C. under vacuum and then subjected to primary drying, followed by secondary drying above room temperature until complete removal of water and solvent (less than 0.5% by weight), as described in example 1. At the end of the freeze-drying process, the headspace of the vials is saturated with a 35/65 mixture of C₄F₁₀/N₂ and the vials are stoppered and sealed. A thermal post-treatment, as described in WO2020/229642 was performed by heating the vials at about 38° C. for about 16 hours.

The vials were then divided in different groups of about 3 vials each.

The first group (C2, comparative) was kept as such, while in the other groups the headspace of the vials was replaced with the following perfluoro olefins or mixtures of perfluoro olefin and nitrogen as defined in table 2.

TABLE 2
Composition of headspace in vials

| | Composition of headspace |
|---|---|
| Preparation | (vol) |
| C2 | 35% C₄F₁₀ + 65% N₂ |
| 2a | 100% C₃F₆ |
| 2b | 100% 2-C₄F₈ |
| 2c | 50% 2-C₅F₁₀ + 50% N₂ |
| 2d | 35% 2-C₅F₁₀ + 65% N₂ |
| 2e | 20% 2-C₅F₁₀ + 80% N₂ |
| 2f | 35% 1-C₅F₁₀ + 65% N₂ |
| 2g | 10% 1-C₆F₁₂ + 90% N₂ |
| 2h | 20% 1-C₆F₁₂ + 80% N₂ |
| 2i | 20% m-2-C₆F₁₂ + 80% N₂ |
| 2j | 35% m-2-C₆F₁₂ + 65% N₂ |

### Example 3

Preparation and Characterization of Suspensions of Gas-Filled Microvesicles (Suspensions C1 and 1a-10)

Suspensions of gas-filled microvesicles were obtained from the preparations illustrated in example 1 by redispersing the respective freeze-dried product in 5 mL of saline.

The various suspensions of gas-filled microvesicles were characterized for their size-distribution, total number of microvesicles and total amount of gas entrapped in the microvesicles of the suspension by using Coulter counter Multisizer™3 (aperture 30 μm-size range 0.7 to 18 μm)

The resistance to pressure of gas-filled microvesicles was measured by using an in-house developed pressure nephelometer. Briefly, the diluted microvesicles suspension was introduced into a spectrophotometer sample cell (airtight and connected to a pressurization system). The optical density (absorbance at 700 nm) of the suspension is continuously recorded while linearly increasing the pressure applied to the sample in the cell from atmospheric pressure (760 mmHg, 101.3 kPa,) to an over pressure of two bars (2280 mmHg, 303.9 kPa), at a rate of about 4 mmHg/s (533 Pa/s).

The Pc50 parameter (“critical pressure”) characterizing each suspension identifies the overpressure (with respect to atmospheric pressure) at which the absorbance of the microvesicles suspension drops to half of its initial value.

The suspensions prepared from the various preparations of Example 1 showed substantially similar concentrations, size-distributions and gas contents. However, the microvesicles comprising the selected perfluoro olefins (in particular perfluoro pentene) showed a substantial increase in pressure resistance when compared to comparative preparation 1. On the other side, preparations where the gas is perfluoro propene show a relatively lower resistance to pressure, as illustrated in table 3.

TABLE 3
Characteristics of gas-filled microvesicles

| | | Pressure |
|---|---|---|
| | Composition | resistance |
| Preparation | of headspace | (Pc50, mmHg) |
| C1 (comp) | 100% SF₆ | 308 |
| 1a | 100% C₃F₆ | 122 |
| 1b | 100% 2-C₄F₈ | 322 |
| 1c | 100% 1-C₄F₈ | 287 |
| 1d | 50% C₃F₆ + 50% N₂ | 125 |
| 1e | 50% 2-C₄F₈ + 50% N₂ | 341 |
| 1f | 50% 2-C₅F₁₀ + 50% N₂ | 511 |
| 1g | 20% C₃F₆ + 80% N₂ | 121 |
| 1h | 20% 2-C₄F₈ + 80% N₂ | 306 |
| 1i | 20% 1-C₄F₈ + 80% N₂ | 272 |
| 1j | 20% 2-C₅F₁₀ + 80% N₂ | 458 |
| 1k | 20% 1-C₅F₁₀ + 80% N₂ | 376 |
| 1l | 10% 1-C₆F₁₂ + 90% N₂ | 239 |
| 1m | 20% 1-C₆F₁₂ + 80% N₂ | * |
| 1n | 20% m-2-C₆F₁₂ + 80% N₂ | 304 |
| 1o | 35% m-2-C₆F₁₂ + 65% N₂ | * |

* suspension not sufficiently stable to measure pressure resistance

As inferable from the above table, preparations with perfluoro pentene, and particularly with perfluoro-2-pentene show a particularly improved resistance to pressure as compared to similar preparations with SF₆. Also, preparations with perfluoro pentenes show a higher resistance to pressure with respect to preparations comprising similar compositions of other perfluoro olefin gases having either lower or higher molecular weights. Furthermore, it can be observed that preparations with perfluoro-2-pentene unexpectedly show a higher pressure resistance with respect to similar preparations with perfluoro-1-pentene.

### Example 4

Preparation and Characterization of Suspensions of Gas-Filled Microvesicles (Suspensions C2 and 2a-21)

Suspensions of gas-filled microvesicles were obtained from the preparations illustrated in example 2 by redispersing the respective freeze-dried product in 5 ml of saline.

The various suspensions of gas-filled microvesicles were characterized according to the procedures illustrated in example 3. The suspensions showed substantially similar concentrations, size-distributions and gas contents. Table 4 shows the measurements of the pressure resistance of each preparation.

TABLE 4
Characteristics of gas-filled microvesicles

| | Composition of | Pressure resistance |
|---|---|---|
| Preparation | headspace (vol) | (Pc50, mmHg) |
| C2 | 35% C₄F₁₀ + 65% N₂ | 623 |
| 2a | 100% C₃F₆ | 151 |
| 2b | 100% 2-C₄F₈ | 295 |
| 2c | 50% 2-C₅F₁₀ + 50% N₂ | 464 |
| 2d | 35% 2-C₅F₁₀ + 65% N₂ | 418 |
| 2e | 20% 2-C₅F₁₀ + 80% N₂ | 369 |
| 2f | 35% 1-C₅F₁₀ + 65% N₂ | 319 |
| 2g | 10% 1-C₆F₁₂ + 90% N₂ | 230 |
| 2h | 20% 1-C₆F₁₂ + 80% N₂ | * |
| 2i | 20% m-2-C₆F₁₂ + 80% N₂ | 304 |
| 2j | 35% m-2-C₆F₁₂ + 65% N₂ | * |

* suspension not sufficiently stable to measure pressure resistance

As inferable from the above table, while the pressure resistance of microvesicles comprising a perfluoro olefin was generally lower than the one of microvesicles comprising a C₄F₁₀, the values are nevertheless well within the acceptable values for the diagnostic/therapeutic use of such microvesicles. Similarly to the results of the previous example, preparations with perfluoro pentenes show a higher resistance to pressure with respect to preparations comprising similar compositions of other perfluoro olefin gases having either lower or higher molecular weights. Furthermore, preparations with perfluoro-2-pentene unexpectedly show a higher pressure resistance with respect to similar preparations with perfluoro-1-pentene.

1. A suspension of gas-filled microvesicles, said gas-filled microvesicles comprising an outer layer of stabilizing material surrounding a gaseous core, wherein said gaseous core comprises a perfluoro pentene.

2. The suspension according to claim 1 wherein said perfluoro pentene is selected from perfluoro-1-pentene, perfluoro-2-pentene, or mixtures thereof.

3. The suspension according to claim 2 wherein said perfluoro pentene is perfluoro-2-pentene.

4. The suspension according to claim 1, wherein said perfluoro pentene is admixed with nitrogen.

5. The suspension according to claim 4 wherein said perfluoro pentene is from 10% to 70% by volume.

6. The suspension according to claim 4 wherein said perfluoro pentene is from 10% to 60% by volume.

7. The suspension according to claim 1, wherein said stabilizing material comprises a phospholipid.

8. The suspension according to claim 7, wherein said stabilizing material further comprises a fatty acid.

9. A vial comprising:
a precursor of gas-filled microvesicles in the form of a freeze-dried product comprising a stabilizing material capable of forming a stabilizing layer for said microvesicles and a freeze-drying protecting component; and
a gaseous component comprising a perfluoro pentene.

10. The vial according to claim 9 wherein said perfluoro pentene is selected from perfluoro-1-pentene, perfluoro-2-pentene, or mixtures thereof.

11. The vial according to claim 10 wherein said perfluoro pentene is perfluoro-2-pentene.

12. The vial according to claim 9, wherein said perfluoro pentene is admixed with nitrogen.

13. The vial according to claim 12 wherein said perfluoro pentene is from 10% to 70% by volume.

14. The vial according to claim 12 wherein said perfluoro pentene is from 10% to 60% by volume

15. The vial according to claim 9, wherein said stabilizing material comprises a phospholipid.
